(19) United States
(12) Patent Application Publication
(10) Pub. No.: US 2017/0065771 A1
(43) Pub. Date: Mar. 9, 2017

(54) PISTON CLOSURES FOR DRUG DELIVERY CAPSULES

(71) Applicant: ZOGENIX, INC., Emeryville, CA (US)

(72) Inventors: Geoff NEWELL, Newmarket (GB); Brooks M. BOYD, Berkeley, CA (US); Philip Justus WUNDERLE, III, El Cerrito, CA (US)

(73) Assignee: ZOGENIX, INC., Emeryville, CA (US)

(21) Appl. No.: 15/357,674

(22) Filed: Nov. 21, 2016

Related U.S. Application Data
(63) Continuation of application No. 13/867,762, filed on Apr. 22, 2013.
(60) Provisional application No. 61/637,008, filed on Apr. 23, 2012.
(60) Provisional application No. 61/779,761, filed on Mar. 13, 2013.

(51) Int. Cl. A61M 5/315; A61K 9/48; A61M 5/30; A61M 5/20; A61M 5/28

(52) U.S. Cl. CPC A61M 5/31513; A61M 5/2053; A61M 5/2046; A61M 5/28; A61M 5/30; A61K 9/4816; A61M 2005/2013

(57) ABSTRACT

A drug capsule and a method for making a drug capsule for a drug delivery device, such as an auto injector or needle-free injector, with improved stability and container closure integrity. The injector comprises a drug capsule sealed by a piston fabricated from PTFE modified by the inclusion of a co-polymer of PPVE, preferably in an amount less than 1% by weight, resulting in better performance while the device is stored and subjected to temperature cycling.

### FIELD OF THE INVENTION

The present invention relates to a piston comprised of polytetrafluoroethylene (PTFE) modified with perfluoro(propyl vinyl ether) (PPVE) to form a copolymer. The piston is used in a drug delivery system such as a pre-filled syringe, an auto-injector, or especially a needle-free injector, for delivery of liquid formulations contained in drug capsules. Delivery is preferably by needle free injection, wherein the piston is both a mechanical system for delivery and a closure seal for the formulation container. The material used to construct the piston is selected so that the piston will have properties such that the container closure system maintains integrity over the range of storage and stability testing temperatures expected for the device.

### BACKGROUND OF THE INVENTION

Many drugs need to be delivered outside of the physician's office, for example due to the need for acute treatment or frequent administration, such as continuously, daily, twice daily, four times daily, weekly, bi-weekly, or monthly. For this reason, the drugs often need to be delivered by someone who is not a skilled medical service provider such as the patient or a family member of the patient. Passive systems such as oral dosage forms, simple nasal sprays, or passive transdermal patches can be used, but auto-injectors, automated pumps, bolus injectors, active transdermal systems, or sophisticated pulmonary delivery systems are often preferred for these products, because of features chosen from their relative ease of use, high dose control and repeatability, ability to titrate the dose or control infusion rate, compliance monitoring features, dose reminders, etc.

Oral drugs have the advantage that they are easy to self administer and are generally accepted by the patient. However, many drugs, especially peptide and protein drugs, have very limited oral bioavailability, due to digestion and first pass liver metabolism. Additionally, absorption following oral delivery is delayed, with time to peak plasma concentrations (Tₘₐₓ) of ˜40 minutes or longer. Thus, a dosage form and/or drug delivery device that is easy and fast to self administer can be crucial for acute, debilitating conditions, for example migraine and cluster headache, hypoglycemia, hyperglycemia, seizure, allergic reaction including anaphylaxis, drug overdose, acute asthma, exposure to warfare agents such as toxins or bioweapons, acute pain, erectile dysfunction, snake, insect, and spider bite, heart conditions, fainting, anxiety, psychotic episodes, insomnia, leg cramps, and other acute conditions.

Many patients and unskilled care givers have difficulties administering drugs, including but not limited to inability of lack of desire to follow complex directions, fear of self administration or administering drug to another, etc. Ensuring treatment compliance and proper delivery can be problematic, especially with complex systems that require filling, reconstitution, and other preparation steps. Thus there is a great advantage to a delivery system that is easy and quick to use, with minimal steps required for preparation and delivery, such as a prefilled syringe, an auto-injector including but not limited to a prefilled autoinjector or an autoinjector with a prefilled, replaceable drug capsule, prefilled pump, a pump with a replaceable prefilled drug capsule, a prefilled transdermal system, a transdermal system with a replaceable drug capsule, a prefilled inhaler, or an inhaler with a preplaceable drug capsule. A preferred drug delivery system is a prefilled, single dose, disposable autoinjector, more preferably a needle free injector. The drug delivery system should require a minimal number of steps for preparation and delivery, preferably less than ten steps, more preferably less than five, most preferably three, two, or one step.

Many pharmaceutically active compounds need to be delivered parenterally by injection or infusion for reasons of low bioavilability when delivered via other routes such as oral, buccal, nasal, pulmonary, or transdermal, or the need for more rapid onset than can be achieved by other routes. Most injectors, including prefilled syringes and autoinjectors, comprise an injection needle. Many patients, however, are needle-averse or suffer from needle-phobia. In addition, injectors with needles entail danger of needle stick injury and cross contamination, and require special sharps and biohazard disposal systems which are in general not available outside of hospitals, laboratories, or doctors offices. In addition, it is a problem that patients may need to be trained to self administer an injection, although for some indications the number of injections they would self administer is only a few. In addition, a needle and syringe in general needs to be filled, and for some formulations dried drug requires reconstitution, which further complicates self administration and reduces compliance. These issues often rule out the possibility of treatment in a home setting, either self treatment or by a relatively un-trained care giver such as a family member. The inability to dose at home can lead to higher costs of therapy, delay in treatment, reduced compliance, reduced comfort, and potential exposure to hospital acquired infections.

In addition, in a hospital, clinic, or doctor's office setting, there is a large advantage to easy to use drug delivery devices and dosage forms, to reduce cost, time, training requirements, risk of injury, and dosing errors. Therefore, there is a significant need for simple, easy to use drug capsules for such systems as pole mounted and table top pump systems, injectors, aerosol delivery systems, and the like.

Some drug delivery systems have drug capsules which are factory prefilled with a liquid formulation, to minimize the amount of preparation required for delivery. Alternatively, capsules may be multi compartment and contain a powdered formulation and a diluent for reconstitution. These capsules can either be integrated into a device which is disposed of when the formulation is exhausted, or multiple capsules can be supplied with a durable device to which they are integrated prior to use, and the capsule is disposed of after delivery. Drug capsules may comprise a polymer or metal, but preferably have a glass component in direct contact with the formulation, more preferably a borosilicate glass component.

Drug capsules which are pre-filled function as the primary container closure system which ensures stability and sterility of the formulation during storage. The drug capsule components must be made of materials that are compatible with the formulation when in contact during storage, and not cause degradation of the formulation components. They also must not leach unacceptable levels of materials into the formulation during storage. The materials and design of the drug capsule must isolate the formulation during storage, not allowing ingress of contaminants, air, water vapor, bacteria, or viruses. The materials and design of the drug capsule must also ensure that there is no egress of formulation components, especially liquid components such as water for injection. The stability and sterility of the formulation must in general be maintained for storage periods of 6 months, preferably for 1 year, more preferably for 2 years, still more preferably for a period of 3 years or more.

In many prefilled drug delivery systems or dosage forms, the drug capsule functions as a syringe. The capsule of this type of injector will have a polymer, metal, or preferably glass syringe body. The syringe body will have in general an exit orifice leading to, for example, a needle, a system for connecting a needle such as a luer fitting, a needle free injector injection orifice, an aerosol generator, a transdermal applicator, an infusion set, a secondary dose chamber for multidose systems, or the like. The syringe body will also in general be sealed in another region by a stopper which also functions as the syringe piston during delivery.

Prefilled drug capsules must be tested to demonstrate that they will provide adequate stability and sterility of the formulation during storage. This testing is called container/closure integrity testing. They must also be tested to ensure that capsule components in contact with the formulation have sufficient low levels of components that will leach into the formulation that will leach into the formulation during storage, generally called leachable and extractable testing. Often testing is done at elevated or reduced temperatures, to ensure that container closure integrity is maintained over the range of temperatures expected in the storage of the device. Elevated temperature testing is also done to estimate the effects of longer term storage, called accelerated stability testing. Temperature testing may also be done by cycling the temperature of the drug capsule between predetermined high and low temperatures for a predetermined number of cycles, and holding the capsule at the high and low temperature for predetermined times. This type of testing is referred to as temperature cycling or thermal cycling. Temperature testing is often combined with drug stability, dye ingress, water vapor transmission rate, microbial challenge, or other tests to demonstrate stability and sterility. Thus the drug capsule components, including syringe body, piston, and exit orifice sealing feature(s), must be designed and made of components that will maintain container closure integrity at elevated temperatures, reduced temperatures, and during temperature cycling.

The drug capsule, and especially the piston and syringe body of a syringe type drug capsule, are subject to very high stresses to ensure a sufficient seal during storage. These stresses, especially of the piston, are in general even higher during piston insertion. In general, the index of thermal expansion of the piston and syringe body of a prefilled syringe will be different, which can further increase stresses during elevated temperature or thermal cycling. This problem is especially acute when the drug capsule comprises borosilicate glass. Borosilicate glass is a preferred material because of its wide application in drug containers and laboratory glassware. Borosilicate glass has a very low index of thermal expansion, greatly reducing its propensity to break when exposed to elevated temperatures and temperature gradients. However, this property of low thermal expansion can lead to high stresses at elevated temperatures if other components, such as a syringe piston, do not have similarly low thermal expansion coefficients. When a component such as a piston is fabricated from a polymer, such as rubber, plastic or PTFE, high stresses can lead to permanent deformation due to yield, or over longer periods, creep. This can lead to a significant problem during temperature changes during storage or testing. For example, if a syringe piston yields or creeps when in a borosilicate glass capsule at high temperature, when the temperature is subsequently reduced, the piston may no longer have sufficient sealing properties in the syringe body, leading to loss of container closure integrity. This problem is especially acute during thermal cycling, when the drug capsule is exposed to elevated and then reduced temperatures, as creep or yield at the elevated temperature is more likely to lead to loss of container closure integrity at the reduced temperature. It is an additional problem that the reduction in sealing combined with thermal cycling can cause the piston to move over time in the syringe body, potentially impacting dosing performance and dose uniformity.

Thus it can be seen that the material and design of prefilled syringe drug capsule components much be selected very carefully to ensure container closure integrity and injector performance over shelf life and during testing.

Some issues are particularly acute in the context of elevated viscosity formulations, including but not limited to controlled release formulations, and formulations of biologic drugs, such as Monoclonal AntiBodies (MABs). Elevated viscosity leads to many delivery difficulties, such as high required hand strength for a needle and syringe, long delivery times, and additional pain and fear associated with a large bore needle. Thus there is a need to deliver these compounds without a needle, preferably in a rapid, automated fashion using a system that does not require filling, reconstitution, or other complex procedures.

One particularly preferred drug delivery device is the needle free injector. Needle free injectors have many advantages over other drug delivery systems, particularly for home use. They have advantages similar to needle injectors, such as high bioavailability, rapid onset, and high reproducibility. They also have many of the advantages of other delivery methodologies, such as avoidance of needle phobia, avoidance of needle stick injury, reduced or no pain, and no requirement for sharps disposal.

Needle-free injectors are available using many different types of energy storage. The energy may be supplied by the user, for example where a spring is manually compressed and latched to temporarily store the energy until it is required to actuate the injector. Alternatively, the injector may be supplied having the energy already stored—for instance by means of a pre-compressed spring (mechanical or compressed gas), or by pyrotechnic charge.

Some injectors are intended for disposal after a single use, whereas others have a re-loadable and/or multidose energy storage means and a single or multi-dose medicament cartridge, and there are many combinations to suit particular applications and markets. For the purposes of the present disclosure, the term “actuator” will be used to describe the energy storage and release mechanism, whether or not it is combined with a medicament cartridge. In all cases, it is necessary to arrange for sufficient force at the end of the delivery to deliver the entire dose of medicament at the required pressure.

EP 0 063 341 and EP 0 063 342 disclose a needle-free injector which includes a piston pump for expelling the liquid to be injected, which is driven by a motor by means of a pressure agent. The liquid container is mounted laterally to the piston pump. The amount of liquid required for an injection is sucked into the pump chamber by way of an inlet passage and a flap check valve when the piston is retracted. As soon as the piston is moved in the direction of the nozzle body the liquid is urged through the outlet passage to the nozzle and expelled. The piston of the piston pump is a solid round piston.

EP 0 133 471 describes a needle-free vaccination unit which is operated with carbon dioxide under pressure, from a siphon cartridge by way of a special valve.

EP 0 347 190 discloses a vacuum compressed gas injector in which the depth of penetration of the injected drug can be adjusted by means of the gas pressure and the volume of the drug can be adjusted by way of the piston stroke.

EP 0 427 457 discloses a needle-free hypodermic syringe which is operated by means of compressed gas by way of a two-stage valve. The injection agent is disposed in an ampoule which is fitted into a protective casing secured to the injector housing. The ampoule is fitted on to the end of the piston rod. Disposed at the other end of the ampoule is the nozzle whose diameter decreases towards the end of the ampoule.

WO 89/08469 discloses a needle-free injector for one-off use. WO 92/08508 sets forth a needle-free injector which is designed for three injections. The ampoule containing the drug is screwed into one end of the drive unit, with the piston rod being fitted into the open end of the ampoule. At its one end, the ampoule contains the nozzle through which the drug is expelled. A displaceable closure plug is provided approximately at the center of the length of the ampoule. The dose to be injected can be adjusted by changing the depth of the ampoule. The piston rod which projects from the drive unit after actuation of the injector is pushed back by hand. Both units are operated with compressed gas.

WO 93/03779 discloses a needle-free injector with a two-part housing and a liquid container which is fitted laterally to the unit. The drive spring for the piston is stressed by means of a drive motor. The spring is released as soon as the two parts of the housing are displaced relative to each other by pressing the nozzle against the injection location. Respective valves are provided in the intake passage for the liquid and in the outlet of the metering chamber.

WO 95/03844 discloses a further needle-free injector. It includes a liquid-filled cartridge which at one end includes a nozzle through which the liquid is expelled. At the other end the cartridge is closed by a cap-type piston which can be pushed into the cartridge. A piston which is loaded by a prestressed spring, after release of the spring, displaces the cap-type piston into the cartridge by a predetermined distance, with the amount of liquid to be injected being expelled in that case. The spring is triggered as soon as the nozzle is pressed sufficiently firmly against the injection location. This injector is intended for one-off or repeated use. The cartridge is arranged in front of the spring-loaded piston and is a fixed component of the injector. The position of the piston of the injector which is intended for a plurality of uses is displaced after each use by a distance in a direction towards the nozzle. The piston and the drive spring cannot be reset. The pre stressing of the spring is initially sufficiently great to expel the entire amount of liquid in the cartridge all at once. The spring can only be stressed again if the injector is dismantled and the drive portion of the injector assembled with a fresh, completely filled cartridge.

U.S. Pat. No. 5,891,086 describes a needle-free injector, combining an actuator and a medicament cartridge. The cartridge is pre-filled with a liquid to be injected in a subject, and having a liquid outlet and a free piston in contact with the liquid, the actuator comprising an impact member urged by a spring and temporarily restrained by a latch means, the impact member being movable in a first direction under the force of the spring to first strike the free piston and then to continue to move the piston in the first direction to expel a dose of liquid through the liquid outlet, the spring providing a built-in energy store and being adapted to move from a higher energy state to a lower energy state, but not vice versa. The actuator may comprise trigger means to operate the said latch, and thus initiate the injection, only when a predetermined contact force is achieved between the liquid outlet of the said cartridge and the subject.

In U.S. Pat. No. 3,859,996, Mizzy discloses a controlled leak method to ensure that the injector orifice is placed correctly at the required pressure on the subject's skin at the correct normal to the skin attitude. When placement conditions are met, controlled leak is sealed off by contact pressure on the subject's skin, the pressure within the injector control circuit rises until a pressure sensitive pilot valve opens to admit high pressure gas to drive the piston and inject the medicament.

In WO Patent 82/02835, Cohen and Ep-A-347190 Finger, disclose a method to improve the seal between the orifice and the skin and prevent relative movement between each. This method is to employ a vacuum device to suck the epidermis directly and firmly onto the discharge orifice. The discharge orifice is positioned normal to the skin surface in order to suck the epidermis into the orifice. This method for injection of the medicament into the skin and the injector mechanism are different and do not apply to the present invention because of its unique ampoule design.

In U.S. Pat. No. 3,859,996 Mizzy discloses a pressure sensitive sleeve on the injector which is placed on the subject, whereby operation of the injector is prevented from operating until the correct contact pressure between orifice and the skin is achieved. The basic aim is to stretch the epidermis over the discharge orifice and apply the pressurized medicament at a rate which is higher than the epidermis will deform away from the orifice.

In U.S. Pat. No. 5,480,381, T. Weston discloses a means of pressuring the medicament at a sufficiently high rate to pierce the epidermis before it has time to deform away from the orifice. In addition, the device directly senses that the pressure of the discharge orifice on the subject's epidermis is at a predetermined value to permit operation of the injector. The device is based on a cam and cam follower mechanism for mechanical sequencing, and contains a chamber provided with a liquid outlet for expelling the liquid, and an impact member, to dispel the liquid.

In U.S. Pat. No. 5,891,086, T. Weston describes a needle-free injector that contains a chamber that is pre-filled with a pressurized gas which exerts a constant force on an impact member in order to strike components of a cartridge and expulse a dose of medicament. This device contains an adjustment knob which sets the dose and the impact gap, and uses direct contact pressure sensing to initiate the injection. Further examples and improvements to this needle-free injector are found in U.S. Pat. No. 6,620,135, U.S. Pat. No. 6,554,818, U.S. Pat. No. 6,415,631, U.S. Pat. No. 6,409,032, U.S. Pat. No. 6,280,410, U.S. Pat. No. 6,258,059, U.S. Pat. No. 6,251,091, U.S. Pat. No. 6,216,493, U.S. Pat. No. 6,179,583, U.S. Pat. No. 6,174,304, U.S. Pat. No. 6,149,625, U.S. Pat. No. 6,135,979, U.S. Pat. No. 5,957,886, U.S. Pat. No. 5,891,086, and U.S. Pat. No. 5,480,381, incorporated herein by reference.

A number of biologically-active agents in viscous formulations would benefit from being delivered using the needle-free injector. This group could consist of (but not limited to) anti-inflammatory agents, antibacterial agents, antiparasitic agents, antifungal agents, antiviral agents, anti-neoplastic agents, analgesic agents, anaesthetics, vaccines, central nervous system agents, growth factors, hormones, antihistamines, osteoinductive agents, cardiovascular agents, anti-ulcer agents, bronchodilators, vasodilators, birth control agents and fertility enhancing agents, interferon alpha, growth hormone, osteoporosis drugs including PTH and PTH analogs and fragments, obesity drugs, psychiatric drugs, anti-diabetes, female infertility, AIDS, treatment of growth retardation in children, hepatitis, multiple sclerosis, migraine headaches, and allergic reactions.

The easiest to use drug delivery systems comprise a liquid drug formulation that is prefilled in a drug capsule at the factory. This has the distinct advantage that the patient or care provider does not have to fill the capsule, making it easier and faster to use. Ease of use and rapid delivery can be critical for acute conditions, including but not limited to migraine and cluster headache. However, being prefilled has the disadvantage that the drug formulation container must maintain the required properties of the formulation over the shelf life of the system. These properties include, but are not limited to, the formulation concentration, which can change if water or other carriers are lost to the atmosphere, or if the active pharmaceutical ingredient is absorbed by drug contact surfaces, purity, which can change if the drug is exposed to contaminants from the environment or the drug container components themselves, stability (i.e. the chemical and conformational properties of the drug molecules) which can be adversely affected by contact with poorly chosen drug container materials or contaminants, and sterility, which can be impacted if the drug formulation is exposed to microbial or viral contamination. To maintain these properties, it is essential that the drug formulation container be properly designed, especially in the selection of the materials that are to be in contact with drug formulation during storage. Many materials have been found to be excellent drug contact materials in the sense that they do not impact the purity of the drug formulation by having volatile components that can extract into the formulation, and do not further impact the chemical or conformational properties of the drug due to the drug being stored in contact with them. These materials include glasses, and selected polymers, including but not limited to fluoropolymers such as polytetrafluoroethylene (PTFE). Borosilicate glass is a preferred glass in that it's very low thermal expansion coefficient allows exposure to elevated temperatures, such as may be seen during sterilization, without creating stresses that lead to breakage. For example, ome needle free injectors, such as that described in U.S. Pat. No. 5,891,086, utilize a glass drug container, which is sealed at one end by a PTFE piston.

It is a problem that prefilled drug capsules must maintain their integrity, including a barrier to contamination and water vapor transmission, over the range of temperatures expected during storage of the device. In the case where the drug capsule comprises a piston and syringe body, a difference in thermal expansion coefficient (CTE) between the piston and the syringe body can create a gap at low or high temperature, allowing loss and/or contamination of the formulation. One way to avoid this is to use very soft rubber that is compressed sufficiently such that no gap will occur. However, this soft rubber may not be consistent with other required properties of the piston. Specifically, for needle free injectors of the type described in U.S. Pat. No. 5,891,086, wherein an impact member flies across a gap and subsequently strikes the piston, creating a pressure spike that creates a hole through the skin, the piston must be sufficiently rigid under this high stress condition that a sufficient amount of the energy is transferred to the formulation, a condition that rubber in general does not satisfy. Unfortunately, more rigid materials cannot in general be compressed sufficiently such that container closure integrity is maintained over the range of expected storage temperatures.

PTFE has intermediate properties in that it is soft enough to be inserted into a glass drug container, but rigid enough to transfer energy to the drug formulation. In fact it has the highly beneficial property that it is substantially non-resilient when subjected to a slowly applied force, such as might be seen during insertion into a glass drug container, but is highly resilient when subjected to a rapidly applied force, such as might be seen during a drug delivery event.

If one looks only at the thermal expansion coefficient and compressibility of PTFE, it would be expected that it would be able to maintain a seal in a borosilicate glass drug container over the temperatures to be expected during shelf life, and over the temperatures it would be exposed to during the temperature cycling required for stability testing. However, if one exposes such a system to temperature cycling, for example between 40° C. and 2° C. for 12 hours at each temperature for 30 days (i.e. 30 cycles), one finds that leakage does occur. The reason for this is the large difference in thermal expansion coefficient between PTFE (CTE=1.5×10⁻⁴/° C.) and Borosilicate glass (CTE=3×10⁻⁶/° C.) causes the PTFE at elevated temperatures, already under significant stress after insertion into the drug container, to be exposed to even higher stresses, causing it to yield, and effectively causing it to have a smaller unstressed diameter. When it is subsequently subjected to 2° C., it is no longer compressed enough to maintain a seal.

In general syringes type drug capsules, including but not limited to prefilled syringes or auto-injectors with elastomeric pistons, require the use of silicone oil or some other lubricant to prevent the piston from binding to the inner surface of the syringe barrel. In addition, silicone oil or another lubricant is required to maintain acceptable sliding friction during travel down the barrel. However, the problem with the use of these types of lubricants is that they cause aggregation of many recombinant proteins and biological molecules over time. These aggregates tend to be immunogenic.

### SUMMARY OF THE INVENTION

A drug capsule comprising a piston and syringe body for use in a drug delivery device, including but not limited to an injector, pump, transdermal system, spray system which creates an aerosol for particular types of treatment including but not limited to pulmonary, nasal, dermal, and ocular, preferably a prefilled syringe or an auto-injector, more preferably for use in a needle-free injector system is disclosed. This component may be a substantially cylindrical container comprised of glass which may be ion exchange strengthened borosilicate glass. The cylindrical glass container is open at one end, and the opening is sealed with a piston comprised of materials which allow for maintaining a tight seal between the piston and the glass during temperature changes expected to occur during sterilization, testing, and storage, e.g. 0° C. to 50° C., or 10° C. to 40° C. The piston may be comprised of one or more polymers which polymers may be linked and may form copolymers. The polymers may be polytetrafluoroethylene (PTFE) alone or in combination with perfluoro(propyl vinyl ether) (PPVE). The capsule may be specifically designed for single use, and may be factory filled and sealed with a liquid formulation comprising a pharmaceutically active drug sealed inside using the piston as a seal for an open end of a glass capsule.

An aspect of the invention is a needle-free drug delivery system which comprises a cylindrical syringe body opened at a first end, the body being comprised of a material which does not readily react with the formulation such as a non-reactive high density polymeric material or a glass such as borosilicate glass strengthened with ion exchange. The syringe body may be pre-filled at the factory with a liquid formulation comprised of a pharmaceutically acceptable carrier and a pharmaceutically acceptable drug. The formulation may be specifically designed for injection from a needle-free injector. The system includes a piston which has an external diameter substantially equal to the internal diameter of the syringe body opened at a first end and as such being configured such that the piston seals the first end of the syringe body and prevents the formulation from leaking out of the syringe body. In particular, the piston prevents leakage out of the container over a range of temperature changes which might occur during storage which can include temperature cycling over a range of 0° C. to 50° C. The piston may be comprised of a copolymer. The copolymer may be polytetrafluoroethylene (PTFE) (modified with perfluoro(propyl vinyl ether) (PPVE)).

Another aspect of the invention is a piston sealed drug capsule comprised of a cylindrical syringe body opened at a first end. The body is prefilled at a factory with a single dose of a liquid formulation comprised of a pharmaceutically acceptable carrier and a pharmaceutically active drug. The opened end of the cylindrical syringe body is sealed with a piston comprised of a non-reactive polymeric material such as a copolymer of polytetrafluoroethylene (PTFE) and perfluoro(propyl vinyl ether) (PPVE). The composition of the piston and the internal diameter of the syringe body are comprised of materials and sized so as to maintain the integrity of the formulation inside the syringe body over a period of time of one year or more during temperature cycling which might normally be expected to occur during storage such as temperature ranges of from 0° C. to 50° C.

An object of the invention is to provide a drug capsule for a drug delivery system that enables drug administration in a setting outside of a hospital, clinic, or doctors office, by simplifying the preparation and administration of the drug using the delivery system, reducing fear and anxiety related to drug administration by the patient or unskilled care giver, and reducing the number of steps associated with and the complexity of drug administration.

A further object of the invention is to provide a drug capsule for use in a hospital, clinic, or doctor office setting that reduces costs, improves outcomes, and improves safety by reducing the steps required and the complexity of preparation of a drug delivery system and drug administration.

An objective of the invention is to provide a method for delivering therapeutics that limits the possibility of needle stick and cross contamination, for example with the HIV virus; improves patient compliance; reduces needle phobia, and improves efficacy of drug delivery.

The invention is carried out using a prefilled drug capsule, preferably a drug capsule that functions as a piston and syringe. Preferably the drug may be removably attached to an actuator to for a drug delivery system, whereby the drug capsule can be disposed of and replaced after the drug contents are exhausted. Preferably the drug capsule is permanently attached to a drug delivery system, and the entire system is disposed of when the drug contents are exhausted. The invention can be carried out using any drug delivery methodology whereby the drug formulation is contained in and delivered from the drug capsule, including but not limited to parenteral, dermal, transdermal, buccal, oral, ocular, pulmonary, vaginal, or enteral delivery. Preferably, the invention is carried out using a prefilled syringe or auto-injector, more preferably using a needle free injector. Most preferably, the invention is carried out utilizing a pre-filled, self contained, single use, portable needle free injector.

In a particularly preferred embodiment, the invention is carried out using a needle free injector that is powered by a self contained compressed gas charge, elements of which are described in U.S. Pat. No. 5,891,086 (incorporated by reference in its entirety). This embodiment includes a device for delivering formulations by needle-free injection, for example Sub-Cutaneously (SC), Intra-Dermally (ID) or Intra-Muscularly (IM). An actuator is used in conjunction with a drug cartridge to form a needle-free injector. The cartridge is pre-filled with a liquid to be injected in a subject, the cartridge having at least one liquid outlet and a free piston inward of the liquid outlet in contact with the liquid.

The actuator comprises:

(a) a housing having a forward portion adapted to be connected with the cartridge;

(b) impact member mounted within said housing inward of the forward portion so as to be movable from a first position toward the forward portion to strike the free piston when a cartridge is connected and to continue to move the piston toward the liquid outlet whereby a dose of the liquid is expelled through the liquid outlet in the cartridge;

(c) an element within said housing which prevents movement of the impact member, wherein upon actuation the element allows movement of the impact member. The element may prevent movement by engaging said impact member to prevent movement of the impact member until actuation, or more preferably prevents the energy source from applying force to the impact member. In a preferred embodiment, the energy source is a source of compressed gas, and the element is a gas valve which is opened when the device is actuated. The element may be actuated in many ways including buttons, levers, and the like, but preferably actuation occurs by pressing the liquid outlet against the desired injection site.

The current invention describes various formulations that can be delivered using drug delivery systems comprising a drug capsule, including but not limited to the injector of U.S. Pat. No. 5,891,086. These formulations comprise active ingredients, and may include various polymers, carriers, etc.

An aspect of the invention is a desirable delivery time, especially for high viscosity formulations. Desirable delivery times may include any delivery times wherein the formulation is successfully delivered. Preferred delivery times include those less than the reaction time of a human, for example less than ˜600 ms, more preferably less than 100 ms.

Another aspect of the invention is acceptable pain associated with injection.

Another aspect of the invention relates to alleviation of fear of needles associated with injection of formulations.

Another aspect of the invention relates to the elimination of the danger of needle stick injury and cross-contamination associated with injection of formulations.

Another aspect of the invention relates to the simplification of preparation associated with delivery of formulations, by supplying a pre-filled, single use or multi dose, disposable drug capsules.

Another aspect of the invention relates to the drug release profile associated with injection of high viscosity depot formulation, especially surface eroding systems.

Another aspect of the invention is to supply a piston for use in a drug capsule of a drug delivery device, preferably a drug capsule that functions as a piston and syringe, wherein the piston material is sufficiently lubricious as to not require additional lubricant.

Another aspect of the invention is a container closure system that is compatible with drug formulations, especially comprising at least one active pharmaceutical ingredient chosen from a list including but not limited to: a biologic or nucleic acid, a polynucleic acid, a small molecule therapeutic, a protein, a peptide, and an antibody, preferably a monoclonal antibody.

Another aspect of the invention is to supply a prefilled container closure system comprising a piston and syringe body for a drug delivery device, preferably a prefilled syringe or auto injector, more preferably a needle free injector, wherein the coefficient of thermal expansion of the piston and the syringe body are sufficiently close together that container closure integrity is maintained over the range of temperatures expected during storage and testing of the device.

A further aspect of the invention is to supply a container closure system for an drug delivery device, preferably a prefilled injection device, more preferably a needle free injector, comprising a formulation container that further comprises a glass capsule, preferably a borosilicate glass capsule, sealed by a piston, wherein the piston properties, including but not limited to thermal expansion, yield strength, and recovery after deformation under load (creep), especially at elevated temperatures, are such that ability to maintain container closure integrity is maintained over the range of temperatures expected during storage, sterilization, and testing of the container closure system.

A further aspect of the invention is to supply a prefilled container closure system for a drug delivery device that comprises a glass capsule, sealed by a piston, wherein the piston is naturally sufficiently lubricious that it does not require additional lubricant for insertion, or to deliver the drug formulation.

It is a further aspect of the invention to supply a piston for a needle free injector that is sufficiently compliant that it can be pressed into a syringe body with enough compression to maintain a tight seal over the range of storage and testing temperatures expected, and yet is rigid enough when struck by an impact member that a sufficient fraction of the energy of the impact member is transmitted to the formulation such that a successful needle free injection can be achieved.

It is a further aspect of the invention to provide a container closure system comprising a piston and syringe body for a drug delivery system such that the movement of a piston is sufficiently low over the temperature excursions that are expected during storage and testing as to not impact the functioning of the injector.

It is a further aspect of the invention to provide a method of modifying a PTFE piston of a drug capsule to improve the shelf life, reliability, and container closure integrity of the drug capsule.

It is a further aspect of the invention to provide a piston for a drug capsule, which piston is fabricated from a PTFE material which is modified in such a way that the drug capsule, when the piston is sealingly placed in the drug capsule, preferably in a glass syringe body, more preferably a borosilicate glass syringe body, is better able to maintain container closure integrity and device reliability after the drug capsule is exposed to the range of temperatures and the temperature cycling that is experienced during storage and testing.

It is a further aspect of the invention to provide a piston that seals a container/closure system, said piston having one or more circumferential raised ribs of triangular cross section, preferably of triangular cross section with the top of the triangle where it contacts the syringe body removed to form a frustum, in order to supply high sealing contact sealing pressure while minimizing creep.

These and other objects, advantages, and features of the invention will become apparent to those persons skilled in the art upon reading the details of the formulations and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic diagram of a needle free injector that utilizes the invention.

FIG. 2 shows another embodiment of a needle free injector that utilizes the invention.

FIG. 2a show an embodiment of a latch used in the triggering mechanism of the invention, in the “safe” configuration.

FIG. 2b shows the embodiment of FIG. 2a, in the ready to fire configuration.

FIG. 2c shows the embodiment of FIG. 2a, in the triggered configuration.

FIG. 3 shows another embodiment of a needle free injector that uses the invention.

FIG. 4 shows an embodiment of a drug capsule that can be used with the above and other embodiments of the invention.

FIG. 5 shows the improvement in deformation after an applied load of one preferred material used in the invention vs. PTFE, modified by the inclusion of less than 1% PPVE.

FIG. 6 shows the reduced deformation under load at elevated temperature of a preferred material used in the invention vs. PTFE.

FIG. 7 shows a schematic of the apparatus used to test the integrity of the drug cartridge via dye ingress.

FIG. 8 shows the results of temperature cycling with a PTFE piston previously used in an injector.

FIG. 9 show the results of a measurement of piston movement during temperature cycling utilizing a glass filled PTFE piston previously evaluated for use in an injector.

FIG. 10 shows the results of temperature cycling with a modified PTFE piston used in the invention, where the PTFE has been modified by the inclusion of less than 1% PPVE by weight.

FIG. 11 shows the results of temperature cycling with a modified PTFE piston, modified with the inclusion of less PPVE than that shown in FIG. 9.

### DETAILED DESCRIPTION OF THE INVENTION

Before the present formulations and methods are described, it is to be understood that this invention is not limited to particular devices, components, formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Active Pharmaceutical Ingredient, API, active drug substance, medicament, or the like: A component of a pharmaceutical formulation that is pharmaceutically active and is delivered for a desired effect.

Actuator: A mechanical device for moving or controlling a mechanism or system. An example of an actuator is a lever that a patient uses to ready an autoinjector for delivery. Alternatively, an actuator can refer to the mechanical portion of an drug delivery device that optionally includes a safety that must be set prior to delivery, triggers the device, and ensures the proper pressure profile during delivery. The device may be triggered by many means, such as by pressing a button, pressing the device against a desired injection site, inhaling through the device, etc.

Aggregation: formation of linked molecules held together by Van der Waals forces or chemical bonds.

AUC: Area under the curve, or the integral, of the plasma concentration of delivered drug over time.

Auto-injector: a drug delivery system which is an injector, wherein the important parameters of the dosing, including but not limited to the dose delivered, the rate of delivery, the formulation pressure or pressure profile, the duration of the delivery, the depth of delivery, are controlled automatically by the device without any input from the user during the delivery event. In some cases the user may program certain parameters, such as the dose, into the device prior to delivery. Autoinjectors may be electronically controlled or all mechanical. They may be prefilled, or be filled with formulation by the user prior to the delivery event. Autoinjectors are preferably portable. They may have an external power source such as mains power, but preferably have a self contained power source. Autoinjectors, especially electronic autoinjectors may have additional features such as dosing reminders, compliance monitors, time and date stamps for dosing events, and may include a wired or wireless means of downloading these data. A particularly preferred autoinjector is a portable, self contained, prefilled, single dose disposable, all mechanical needle free injector comprising a pressurized gas power source and a drug capsule comprising borosilicate glass and a modified PTFE piston.

Biodegradable: capable of chemically breaking down or degrading within the body to form nontoxic components. The rate of degradation of a depot can be the same or different from the rate of drug release.

Biologic: A medicinal products created by biological processes (as opposed to chemically). Examples include such as vaccines, blood and blood components, allergenics, [1] somatic cells, gene therapy, tissues, stem cells, immune globulins, and recombinant therapeutic proteins Biologics may be isolated from natural sources such as humans, animals, plants, or microorganism—or may be produced by biotechnology methods.

Borosilicate glass: a type of glass comprising silica and boron that is commonly used in chemical and medical applications. Borosilicate glass has a very low coefficient of expansion (˜3×10⁻⁶) making is less susceptible to breakage when exposed to heat, for example when heat sterilized.

Bulk erosion: The rate of water penetration into the depot exceeds the rate at which the depot is eroded (i.e. transformed into water soluble products)—leading to an erosion process that occurs throughout the entire volume of the depot—true with most hydrophilic polymers used in drug delivery currently.

Carrier: a non-active portion of a formulation which may be a liquid and which may act as a solvent for the formulation, or wherein the formulation is suspended. Useful carriers do not adversely interact with the active pharmaceutical ingredient and have properties which allow for delivery, for example needle free injection. Preferred carriers include water, saline, and mixtures thereof. Other carriers can be used provided that they can be formulated to create a suitable solution and do not adversely affect the drug thereof or human tissue.

Centipoise and centistokes: different measurements of viscosity, which are not just different units. Centipoise is a dynamic measurement of viscosity whereas centistokes is a kinematic measurement of viscosity. The conversion from centistokes and centipoise to s.i. units is given below:
1 cS=0.0001 m²/s 1 cP=0.001 Ns/m²

Coefficient of Thermal Expansion, Thermal Expansion Coefficient, CTE, and the like: The fractional change in a dimension of a material (ΔL/L), per degree C.

Coefficient of Friction: a constant of proportionality relating the normal force between two materials, and the frictional force between those materials. Generally friction is considered to be independent of other factors, such as the area of contact. The coefficient of static friction characterizes the frictional force between to materials when at rest. This force is generally what is required to start relative movement. The coefficient of dynamic friction characterizes the frictional force between to materials that are moving relative to one another. In general, the coefficient of static friction is higher than the coefficient of dynamic friction.

Container Closure, Container Closure System, and the like: A drug container that is designed to maintain sterility and eliminate the possibility of contamination of the drug formulation. For container closure systems that contain aqueous formulations, the container closure system must have sufficiently low water vapor transmission rate such that the concentration of the formulation does not change appreciably over the product shelf life. Preferred materials have sufficiently low extractable materials such that they do not contaminate the formulation. For multi component container closure systems, the interface(s) between the components must be such that liquid carriers, contaminants, including but not limited to microbial and viral contaminants, and gasses such as air cannot appreciably pass through over the shelf life of the system and over the expected temperature range. Container closure system materials that are in contact with the drug formulation must have properties such said contact does not lead to unacceptable levels of degradation of the drug formulation. Preferred materials for container closures include glass, more preferably borosilicate glass, or fluorinated polymers such as polytetrafluoroethylene (PTFE), including modified PTFEs, preferably modified by the inclusion of a PPVE copolymer, more preferably by the inclusion of PPVE in an amount less than 1% by weight.

Container Closure Integrity: The ability of a container closure system to maintain sterility, eliminate the possibility of contamination, and minimize loss of carrier during storage.

Deformation Under Load, Creep, Cold Flow, and the like: Changes in the dimensional properties of a material, especially a polymer, when placed under a load. The load may be externally applied, as when the piston of the current invention is inserted into the glass drug capsule, and may be increased by subjecting the drug formulation container of the current invention to elevated temperatures.

Depot Injection, Depot, and the like: an injection, usually subcutaneous, intravenous, or intramuscular, of a pharmacological agent which releases its active compound in a consistent way over a long period of time. Depot injections may be available as certain forms of a drug, such as decanoate salts or esters. Examples of depot injections include Depo Provera and haloperidol decanoate. Depots can be, but are not always, localized in one spot in the body.

DosePro or Intraject: a single use, prefilled, disposable, needle free injector currently manufactured by Zogenix Corporation. A cartridge is pre-filled with a liquid to be injected in a subject, and having a liquid outlet and a free piston in contact with the liquid, the actuator comprising an impact member urged by a compressed gas spring and temporarily restrained until the device is actuated, the impact member being movable in a first direction under the force of the spring to first strike the free piston and then to continue to move the piston in the first direction to expel a dose of liquid through the liquid outlet, the spring providing a built-in energy store and being adapted to move from a higher energy state to a lower energy state, but not vice versa. The actuator may comprise a trigger means to actuate the device, and thus initiate the injection, only when the device is pressed against the skin. Elements of DosePro are described in U.S. Pat. No. 5,891,086, and additional description and improvements can be found in U.S. Pat. No. 6,620,135, U.S. Pat. No. 6,554,818, U.S. Pat. No. 6,415,631, U.S. Pat. No. 6,409,032, U.S. Pat. No. 6,280,410, U.S. Pat. No. 6,258,059, U.S. Pat. No. 6,251,091, U.S. Pat. No. 6,216,493, U.S. Pat. No. 6,179,583, U.S. Pat. No. 6,174,304, U.S. Pat. No. 6,149,625, U.S. Pat. No. 6,135,979, U.S. Pat. No. 5,957,886, U.S. Pat. No. 5,891,086, and U.S. Pat. No. 5,480,381, incorporated herein by reference. Although many delivery systems and techniques may be used with the current invention, DosePro is the preferred method.

Drug Cartridge, Drug Capsule, and the like: a container closure system utilized in an drug delivery system, and is preferably prefilled and disposable. In a preferred embodiment, the Drug Capsule comprises a glass container, preferably a borosilicate glass container, which forms a syringe body, and is closed on one end by a modified PTFE piston. The glass container comprises at least one delivery orifice, preferably opposite the piston, which is sealed, for example by an end cap, prior to preparation for use. Preferably the glass container is contained in a polymeric sleeve, which comprises a feature such as screw threads for attachment to an actuator. The glass container may be strengthened to avoid breakage upon actuation by ion exchange strengthening. The drug capsule may contain multiple doses, or preferably contains a single dose and is disposed of after a delivery event.

Drug Delivery System, Drug Delivery Device, and the like: a system for delivery of a formulation to an animal or preferably a human subject. Preferred drug delivery systems include a prefilled drug capsule which functions as a container closure system and also comprises a piston and syringe body to deliver the formulation from the drug capsule, either directly to the subject, or to an additional component or subsystem that delivers the formulation. The drug capsule may be disposed of and replaced after the drug is exhausted, or preferably permanently integrated with the actuator, whereby the entire drug delivery system is disposed of after the drug is exhausted. Drug delivery systems may be parenteral, transdermal, pulmonary, buccal, enteral, oral, ocular, vaginal, or deliver by any other route of delivery. Preferred drug delivery systems are prefilled syringes, pumps, or auto-injectors, most preferably the drug delivery system is a needle free injector, preferably a portable, self contained, prefilled, single use disposable needle free injector.

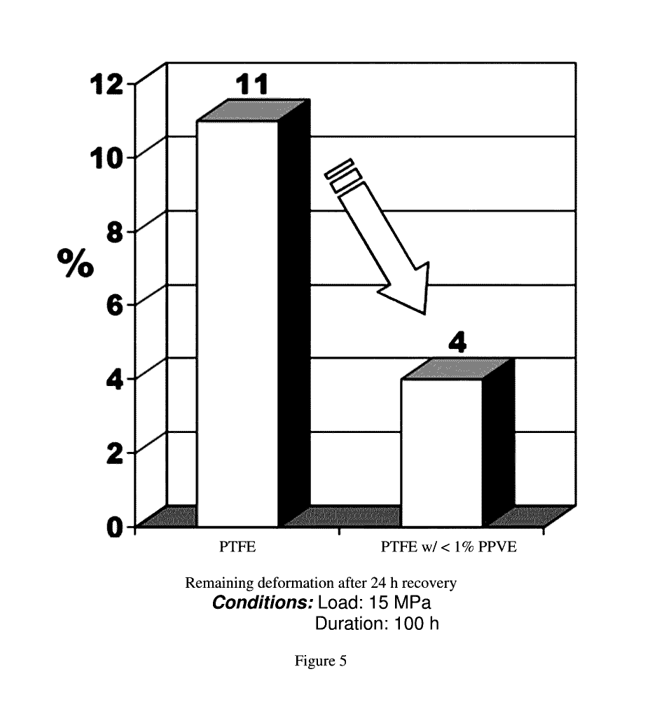

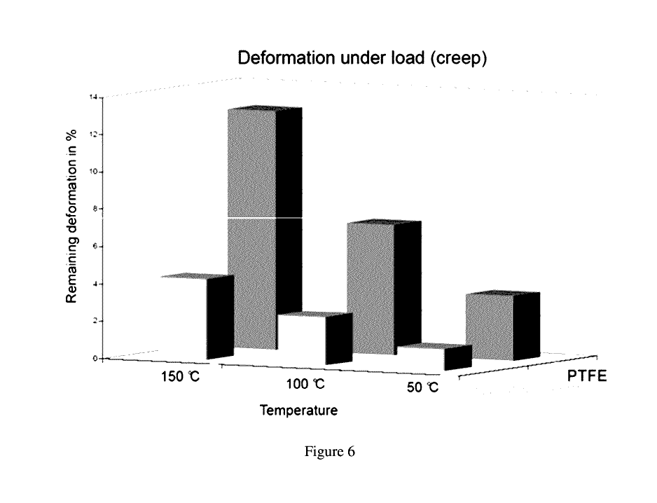

Dye Ingress, Dye Penetration, and the like: A test of container/closure integrity, wherein the drug capsule of the current invention is exposed to a dye, and then inspected to see if any of the dye has penetrated to the liquid formulation. FIG. 6 shows schematically a dye ingress apparatus. This test is preferably performed after temperature cycling in an environmental chamber, wherein the temperature of the drug capsule is cycled up and down in a predetermined manner (see “thermal cycling”)

Excipient: Any substance, including a carrier, added to an active drug substance to permit the mixture to achieve the appropriate physical characteristics necessary for effective delivery of the active drug.

Formulation: Any liquid, solid, powder, or other state of matter that can be delivered from a drug delivery device. Preferred formulations are liquid formulations, including but not limited to solutions, suspensions including nano-suspensions, emulsions, polymers and gels. Formulations include but are not limited to those containing excipients that are suitable for administration to a human, and contain one or more active pharmaceutical ingredients.

Immunogenicity: Immunogenicity is the ability of a substance (an antigen) to provoke an immune response. Aggregated biologic drugs can be immunogenic even when the unaggregated molecule is not immunogenic.

Needle free Injector, Needle-less injector, and the like: a drug delivery system delivers a subcutaneous, intramuscular, or intradermal injection without the use of a hypodermic needle. Injection is achieved by creating at least one high velocity liquid jet with sufficient velocity to penetrate the skin, stratum subcutaneum, or muscle to the desired depth. Needle free injection systems include, but are not limited to, the DosePro® system manufactured by Zogenix Corporation, the Bioject® 2000, Iject or Vitaject devices manufactured by Bioject Medical Technologies, Incorporated, the Mediject VISION and Mediject VALEO devices manufactured by Antares, the PenJet device manufactured by Visionary Medical, the CrossJect device manufactured by Crossject, the MiniJect device manufactured by Biovalve, the Implaject device manufactured by Caretek Medical, the PowderJect device manufactured by AlgoRx, the J-tip device manufactured by National Medical Products, the AdvantaJet manufactured by Activa Systems, the Injex 30 device manufactured by Injex-Equidyne, and the Mhi-500 device manufactured by Medical House Products.

Perfluoropropyl Vinyl Ether, PPVE, and the like: a polymer used in the manufacture of fluoropolymers and other specialty agrochemical and pharmaceutical applications. In the context of the present invention, PPVE is used to modify PTFE to improve its properties for use in injection pistons. Preferably, the PTFE is modified by the inclusion of less than 1% PPVE by weight.

Polytetrafluoroethylene, PTFE, Teflon, and the like: a synthetic fluoropolymer of tetrafluoroethylene. PTFE is most well known by the DuPont brand name Teflon. PTFE is a high molecular weight fluorocarbon solid, consisting wholly of carbon and fluorine. PTFE has one of the lowest coefficients of friction against any solid.

Portable: easily carried by a person, possibly by hand or in a back pack, but preferably in a purse, pocket or the like. A portable drug delivery device had a longest dimension which is less than 30 cm, preferably less than 25 cm, more preferably less than 20 cm, most preferably less than or about 15 cm. Portable drug delivery devices are preferably self contained.

Prefilled: Filled with formulation prior to being received by the end user, i.e. patient or care giver. A drug capsule can be prefilled at a pharmacy, but preferably will be prefilled at a factory prior to being packaged and shipped. Prefilled capsules will require testing to demonstrate they will be able to maintain stability and sterility of the drug formulation over the shelf life, and over the range of storage conditions, especially temperature, that are expected during storage and use. In general, prefilled drug capsules will require testing at elevated temperatures, and temperature cycling.

Prophylaxis: The administration of a drug used to prevent the occurrence or development of an adverse condition or medical disorder.

Self Contained: Including all of the components and functionality required to effect drug delivery. A self contained drug delivery system may be a kit which comprises an actuator and one or a multiplicity of replaceable, prefilled drug capsules, but will not require any additional components. A self contained drug delivery system comprises an energy source, such as a battery, mechanical spring, compressed gas source, chemical reaction, or the like. The energy source may contain enough energy for a multiplicity of drug delivery events, and when exhausted may be replaced, recharged, or the entire device may be disposed of. The energy source may also be energized by the user or care giver prior to delivery, for example a mechanical spring that is compressed but do not require the user to input energy during the delivery event. Preferably, a self contained drug delivery device contains sufficient energy for a single drug delivery event, after which is cannot be re-used, and must be disposed of. Self contained drug delivery systems do not require the use of mains power during the delivery event, although they may comprise rechargeable batteries that recharged using mains power prior to the drug delivery event.

Surface Erosion: The rate of water penetration into the depot is slower than the rate at which the depot is eroded. The depot erodes from the surface before water has penetrated the entire volume of the device.

Specific gravity: ratio of a compound's density to that of water.

Spring: a mechanism capable of storing energy for use in propelling the medicament in the syringe out of the drug capsule, through an optional drug delivery component or sub assembly, and into or onto a body, wherein the force provided by the energy store is proportional to a displacement. This mechanism may be mechanical, e.g. compressible metal component such as a coil spring or Belleville washer stack. Preferably, the mechanism is a compressed gas spring in which the energy is stored, and when released the gas expands.

Strain: the deformation of a body, especially the piston of the current invention, when subjected to an external load. Deformation can be elastic, wherein the body returns to its previous configuration after the external load is removed. It can also be inelastic, wherein the body is permanently changed by the load.

Stress, load, and the like: An applied force or pressure that tends to deform a body, especially the piston of the current invention. See also Strain.

Modified PTFE: PTFE that has been modified to improve its performance, for example when used as a material for injection pistons. Preferably, the PTFE is modified by the inclusion of a perfluoropropyl vinyl ether (PPVE) modifier, more preferably by the inclusion of less than 1% by weight of PPVE. PTFE modified in this way it has lower (<⅓) deformation under load than un-modified PTFE under similar conditions of load and temperature.

Thermal Cycling, Temperature Cycling, and the like: a method of testing properties of a drug delivery system, and specifically the container/closure integrity of the drug capsule, of the current invention wherein the object under test is placed in an environmental chamber and exposed to a prespecified set of temperatures that change over time in a prespecified way. In one embodiment of the test, the inside diameter of the glass capsules and the outside diameter of the pistons are measured, the capsules are assembled and are filled with normal saline, placed in an environmental chamber nozzle down and cycled between 40° C. and 2° C. for 12 hours at each temperature for 30 days (i.e. 30 cycles). Movement of the piston relative to the glass capsule is measured at prespecified intervals. At the end of the test, the capsules are exposed to a dye (see “Dye Ingress” and FIG. 6), and checked for leakage.

Water Vapor Transmission Rate (WVTR)) is the steady state rate at which water vapor permeates through a material or out of a drug capsule. Values are expressed in g/100 in²/24 hr in US standard units and g/m²/24 hr in metric units.

### INVENTION IN GENERAL

In general, the container closure system, or drug capsule of a drug delivery device comprises a cylinder, preferably a right circular cylinder, which forms a syringe body. The syringe body generally comprises one or more outlet orifices. The syringe body is closed on one end by a stopper, which preferably during delivery acts as a piston. The outlet orifice either delivers the drug directly, as when it is a needle free injector injection orifice or an aerosolization nozzle, or it may lead to an additional drug delivery component or sub-system, such as a needle, infusion set, or the like. During storage the outlet orifice(s), or the additional component or subsystem, are sealed by a valve, stopper, end cap or the like. Upon triggering of the drug delivery device, the piston slides down the barrel of the cylinder and forces the formulation out of the exit orifice. It is thus required that the friction between the piston and syringe body be sufficiently low such that the available force is sufficient to achieve delivery. To achieve this, lubricant can be used. However, this lubricant will be in contact with the drug formulation, and can have adverse impact on the stability of the formulation. For example, most standard needle and syringe injectors have a rubber stopper lubricated with oil, such as silicone oil, which can lead to issues such as aggregation of protein drugs and other biologics, potentially causing immunogenicity. Thus it is preferred that the piston be made of a material that is sufficiently lubricious that no additional lubricant is required.

One particularly preferred compound for use in a piston is Polytetrafluoroethylene, or PTFE. PTFE is an excellent material for drug formulation contact, as it is very non-reactive, partly because of the strength of carbon-fluorine bonds. PTFE is also very lubricious, having one of the lowest coefficients of friction against most solids. In general, the use of PTFE for a piston obviates the need for a separate lubricant.

Although plastics, for example polycycloolefin, are used for some syringe bodies, including prefilled injectors, the gold standard material for syringe bodies and other drug contact surfaces is glass, more preferably borosilicate glass. However, it is problem that glass and PTFE have significantly different coefficients of thermal expansion, with PTFE having a fairly high thermal expansion coefficient of approximately 10-16*10⁻⁵/deg C., and borosilicate glass having a much lower coefficient, 0.5*10⁻⁵/deg C. This difference in expansion can lead to loss of container closure integrity upon a reduction in temperature. For example, a 10 degree reduction in temperature would lead to a 10 μm difference in contraction for a 1 cm PTFE piston in a borosilicate glass syringe body. Depending on the amount of preload on the PTFE when it is forced into the syringe body and the amount of creep of the PTFE during storage, this differential thermal expansion could lead to as much as a 5 μm gap around the piston, leading to a loss of container closure integrity and potentially leading to loss of sterility, contamination, and/or evaporation of carrier. This problem can be exacerbated if prior to being exposed to low temperature, the drug cartridge is exposed to elevated temperature, for example 40° C. which is often used in accelerated stability and temperature cycling studies. Exposure of the piston to elevated temperature causes it to want to expand. Because it is constrained by the syringe body, this can cause the piston to yield or creep, leading to a smaller effective outside diameter. When subsequently exposed to a reduced temperature, there is a much larger likelihood of loss of container closure integrity.

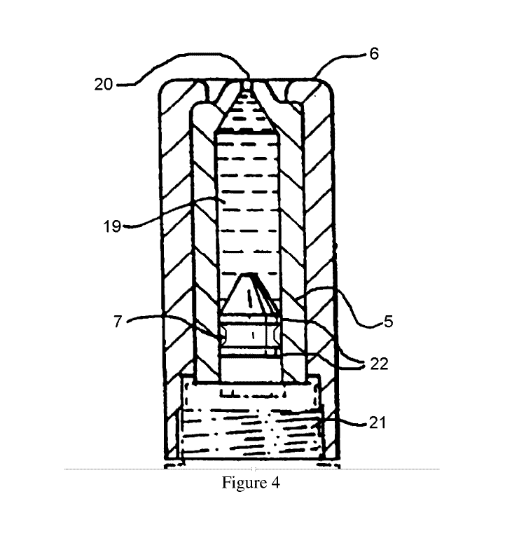

PTFE can be modified to improve its properties for use in pistons for drug delivery systems. Preferably, the modified PTFEs are Tetrafluoroethylene-Perfluoro(Propyl Vinyl Ether) (PPVE) copolymers, comprising less than 1% PPVE by weight. PTFE modified by the inclusion of PPVE have many properties that make them well suited for injection drug delivery piston, including low deformation under load (see FIG. 5), especially at elevated temperatures (see FIG. 6), low coefficient of friction (μ=0.2), low extractables and leachables, high tensile strength (˜40 MPa), wide temperature range (˜200 to 260° C.), low permeation, no water absorption, almost universal chemical resistance, good light and weathering resistance, and high purity.

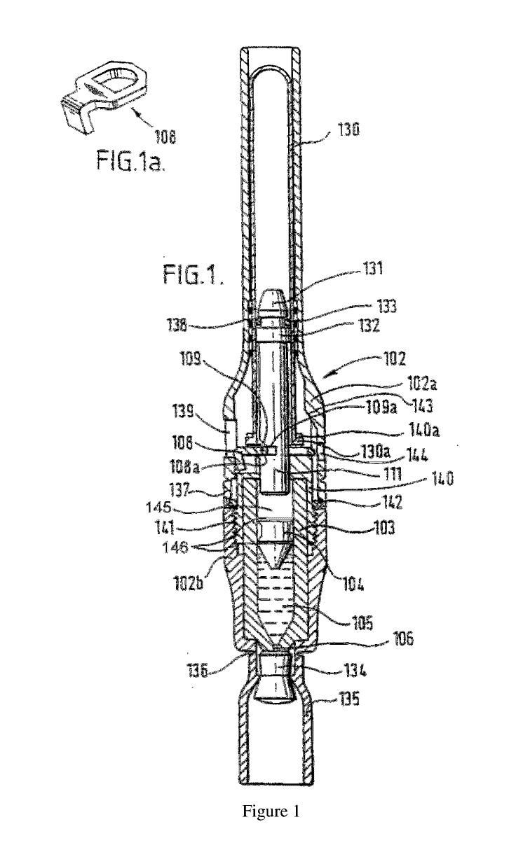

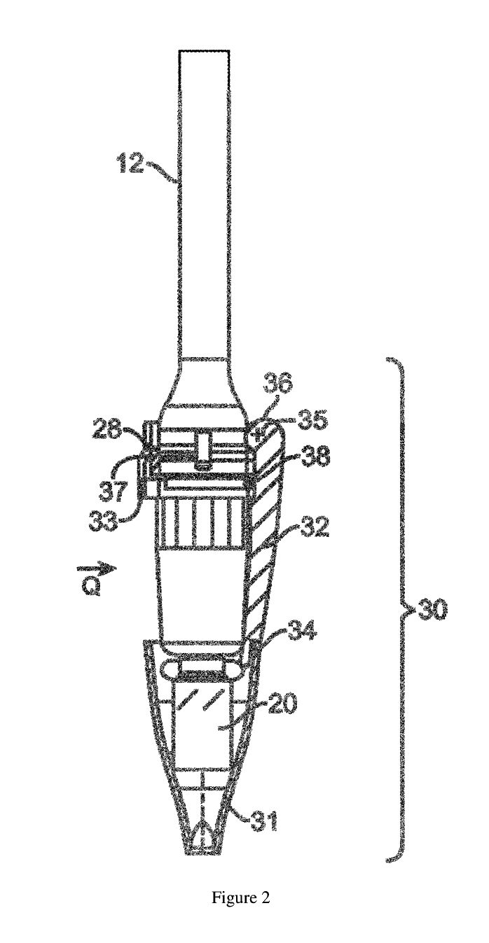

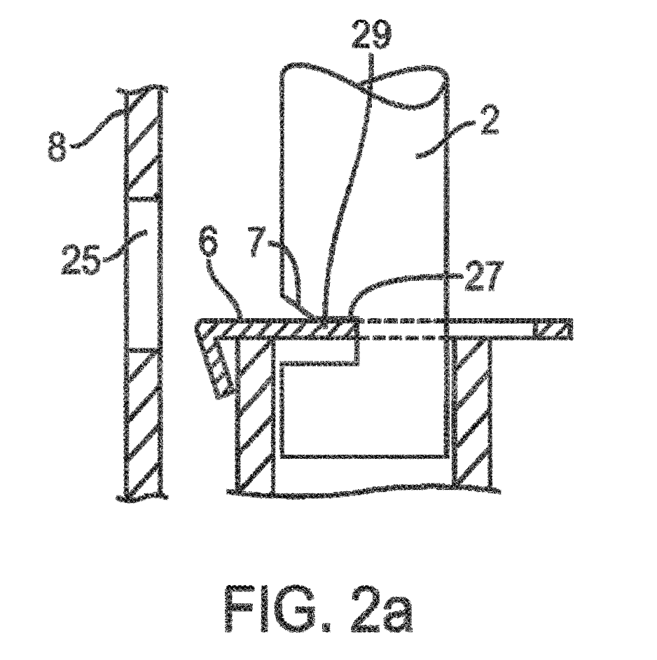

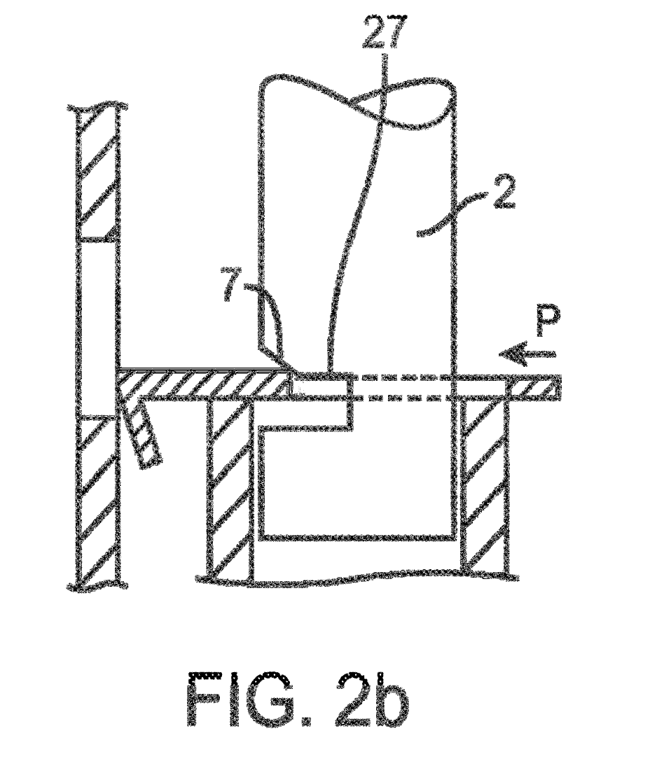

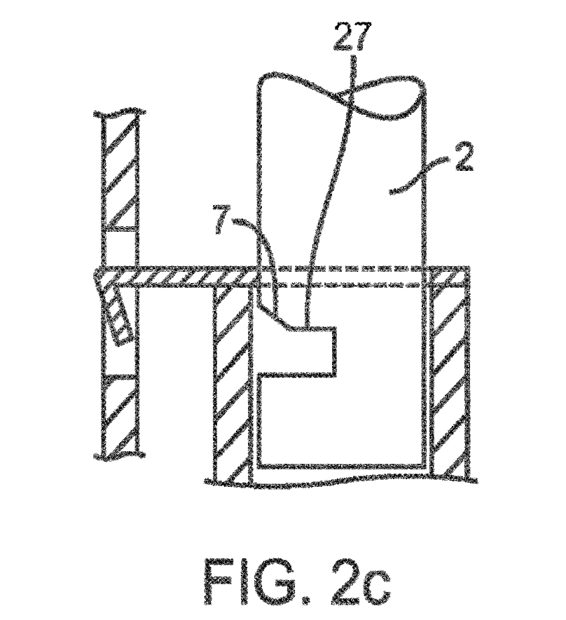

In the needle free injector embodiment of FIG. 1, the injection force is provided by a compressed gas spring. This is in the form of a cylinder 130 which is closed at its upper end and which contains gas, typically air, under a pressure which is typically in the range 5.5 MPa (800 psi) to 20.7 MPa (3000 psi). The cylinder houses a ram 111. The end of the ram 111 has a frusto-conical portion 131 and a flange 132 between which is situated an O-ring seal 133. Prior to use, the ram 111 is held in the illustrated position by a latch 108 engaging in a groove in the ram, the upper surface of the groove forming a cam surface 109. The latch 108 is shown on a larger scale in FIG. 2a. In the position shown in FIG. 1 the latch is unable to move leftwards, because it bears against the inner wall of a sleeve 102.

The lower end of the cylinder 130 has an outwardly directed flange 130a, which enables the cylinder to be held by crimping the flange 130a beneath an outwardly directed flange 140a at the upper end of a coupling 140. The sleeve 102 is formed of an upper sleeve portion 102a within which the cylinder is situated, and a lower sleeve portion 102b. The sleeve portion 102b is connected to the coupling by the inter-engaging screw threads 141 formed on the inner and outer walls of the sleeve portion 102b and coupling 140 respectively.

The injector contains a drug capsule 103 which is preferably glass, more preferably borosilicate glass. drug capsule 103 has a piston 104 slidingly and sealingly located therein, in contact with medicament 105. The properties of piston 104 must be consistent with contact with the formulation 105 over the shelf life of the device, and must ensure stability and sterility of formulation 105 by maintaining a seal over the shelf life and over all temperatures to be seen during storage and during testing. PTFE is a preferred material for piston 104, more preferably a modified PTFE, more preferably PTFE modified by the addition of Perfluoro (Propyl Vinyl Ether) (PPVE) copolymer, most preferably in an amount less than 1%. As considered from the upper end of FIG. 1, piston 104 may comprise a cylindrical portion encircled by a larger diameter sealing portion 146, more preferably with two larger diameter sealing features 146. Larger diameter sealing features 146 function to create the required compression that will maintain sealing over the life of the device without creating too high an insertion force when piston 104 is inserted into glass cartridge 103. Piston 104 further comprises a frusto-conical portion, designed to mate with the lower end of drug capsule 103 at the end of delivery to ensure that essentially all medicament is delivered. The drug capsule 103 has a discharge orifice 106. The orifice 106 is sealed by a resilient seal 134 which is held in place by a seal carrier 135. The seal carrier 135 is connected to the lower sleeve portion 102b by a frangible joint 136.

As a precaution against accidental firing, a removable blocking element 137 is provided between the lower part of the upper sleeve portion 102a. The lower edge of blocking element 137 bears against lower sleeve portion 102a. The function of blocking element 137 is to inhibit relative movement of the upper and lower sections, and thus inhibit triggering of the device, until blocking element 137 is removed. Blocking element 137 may be a tear off band, but is preferably a separate element that is removed by radial displacement.

An annular space 138 is formed in the inside wall of the sleeve 102, where the sleeve is adjacent the cylinder 130, and the space is filled with a damping grease (indicated diagrammatically by a succession of black bands), so that the grease is in intimate contact both with the sleeve 102 and the cylinder 130. It should be noted that although a defined annular space is convenient from the point of view of providing a particular location for the grease, it could be omitted and the grease simply smeared over all or part of the outside of cylinder 130 and/or inside of sleeve 102.

When the embodiment of FIG. 1 is to be operated, the user snaps off seal carrier 135 at frangible joint 136, which takes seal 134 with it and exposes orifice 106. The user then removes blocking element 137, and grasping the upper part of sleeve 102 urges the orifice against the substrate (e.g. the user's own skin) which is to be injected. This moves upper sleeve portion 102a downwardly, with respect to lower sleeve portion 102b. This brings aperture 139 in the wall of upper sleeve portion 102a into alignment with latch 108, which is thus able to move sideways into aperture 139 under the influence of the force of the gas within cylinder 130 acting on latch 108 via cam surface 109 formed in ram 111. The injector is thus caused to fire. The resulting recoil is damped by the damping grease.

FIG. 2 illustrates an embodiment of the needle-free injector with setting means 30 for disengaging the blocking element 38. In this figure, the means for disengaging the blocking element 38 comprises cap 31 enclosing, and holding rigidly, seal carrier 20; lever 32; and collar 33. The lever contains lip 34 at the far end, over which cap 31 is positioned. This ensures that lever 32 cannot be moved before the outer cap 31 is removed, which in turn ensures that the user cannot move the latch or disengage the safety mechanism until the cap has been removed. This is important because if blocking element 38 can be removed before removing cap 31, as is possible in the embodiment shown in FIG. 1, the act of removing cap 31 can cause the device to fire. Lever 32 is pivoted around pivot axis 35, with the pivoted surface in contact with injector being a cam surface 36. The force required to pivot lever 32 is in the range from about 2N to about 30N. Collar 33 contains pin 37 which extends into the device through opening 28 in upper sleeve 12 to impinge on the far side of latch 6. The force required to move latch 6 is in the range from about 20N to about 120N. To stop the upper sleeve section 12 moving with respect to lower sleeve section 13, there is blocking element 38 between the upper and lower sleeves, which form part of collar 33. Blocking element 38 takes the place of the tear off band of the embodiment shown in FIG. 1.

To deliver the device contents, cap 31 is removed, exposing injection orifice 18. With outer cap 31 removed, lip 34 is exposed, enabling lever 32 to rotate about the pivot axis 35. Only when the outer cap 31 is removed can lever 32 be rotated. At this point latch 6 is on flat (non-camming) surface 27 of ram 2, as shown in FIG. 2a. As lever 32 rotates, cam surface 36 forces collar 33 to move in the direction Q, pushing pin 37 against latch 6. When lever 32 has rotated through a complete cycle, approximately 180 degrees, latch 6 moves to the second position, onto ram camming surface 7, as shown in FIG. 2b. Blocking element 38 no longer restricts the movement of upper sleeve 12 with respect to lower sleeve 13 and the device can trigger as described above.

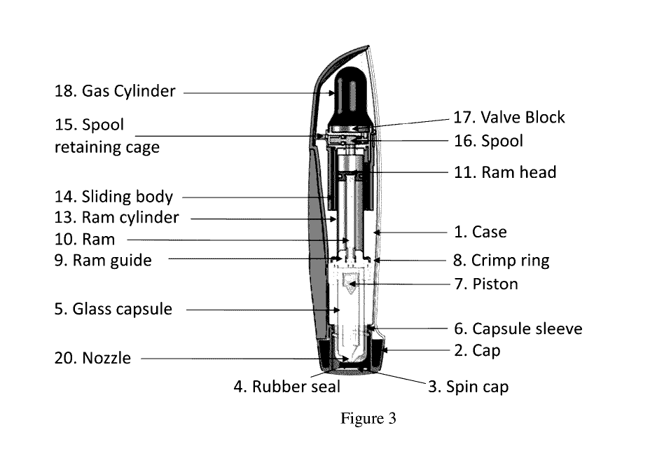

FIG. 3 shows another embodiment of the injector device. In this embodiment, the latch of the previous embodiments is replaced by a spool valve comprising spool 16, valve block 17, and spool retaining cage 15. The operation of this embodiment is as follows: The user removes cap 2, which also removes rubber seal 4 and spin cap 3. Spin cap 3 is provided to ensure that the act of screwing cap 2 onto capsule sleeve 6 doesn't create stresses in rubber seal 4, which can lead to loss of seal. Cap 2 is threaded onto both capsule sleeve 6 and case 1, ensuring that as cap 2 is removed capsule sleeve 6 is biased downward, preventing accidental actuation. Nozzle 20 is then pressed against the desired injection site. This causes the internal components to move upward relative to case 1, sliding body 14, and spool retaining cage 15. When the motion is sufficient, spool 16 is forced into spool retaining cage 15 by the pressure of the gas in gas cylinder 18, allowing the gas to pressurize ram head 11, and the injection proceeds as above.

All of the embodiments in FIGS. 1-3 have in common a drug capsule like that shown in FIG. 4, which can be used with many types of drug delivery systems. The drug capsule comprises a syringe body 5 that is preferably comprised of glass, more preferably comprised of borosilicate glass. Syringe body 5 is contained within capsule sleeve 6. Syringe body 5 is sealed on one end by piston 7, forming a reservoir for drug formulation 19 which is preferably a liquid drug. Piston 7 comprises larger diameter sealing ribs 22. At the opposite end of capsule 5 from piston 7 is outlet orifice 20, which forms the liquid injection jet in the case of needle free injection, can be an aerosolization nozzle in the embodiment where the drug delivery system is a aerosol drug delivery system, or may lead to an additional drug delivery component or sub-assembly such as a needle, infusion set, transdermal technology, or the like. A single outlet orifice is shown in FIG. 4, but the capsule may comprise 2, 3, 4, or more outlet orifices. In the case of the outlet orifice being aerosolization nozzle, the system may comprise more than 100 or more than 1000 outlet orifices. Prior to injection, injection orifice 20 is closed by a seal (not shown). Threads 21 are provided to facilitate attachment to an actuator, such as those disclosed in FIGS. 1-3 or similar systems appropriate to the rate and force required for other delivery methodologies. Sealing ribs 22 function to create the required compression that will maintain sealing over the life of the drug capsule and the temperatures the drug capsule will be exposed to during storage, sterilization, and/or testing, without creating too high of an insertion force when piston 104 is inserted into glass syringe body 5. Sealing ribs 22 have a triangular shape, or preferably a triangular shape with the vertex in contact with the syringe body 5 flattened or truncated to form a frustum. This shape serves to focus the stress into the contact zone with syringe body 5, enabling sealing ribs 22 to maintain the contact pressure at the interface with syringe body 5 while maintaining a lower shear stress in the surrounding material. The high stress contact area is encapsulated by the surrounding material of sealing ribs 22 at a lower stress as the distance from syringe body 5 increases, creating essentially compressive stress at the contact region, making this region not subject to creep.

Use of a prefilled drug delivery device, has many benefits over non prefilled devices such as a standard needle and syringe, including:
No need to draw formulation into the drug capsule prior to use
Fewer steps
Simpler instructions
Minimal amount of equipment required (especially important for acute indications wherein the injection system must be carried around by the user.)
Fast administration
Improved patient compliance
Improved disease outcomes.

Self contained drug delivery devices systems are preferred as the energy for the delivery comes from the device rather than the patient or caregiver that is administering the medication. This can be very important, for example, in the delivery of high viscosity formulations that require high hand strength and long delivery times with a standard needle and syringe.

Prefilled drug delivery systems are preferred as they require fewer or no steps to prepare the device for delivery. This can be very important in the case of self administration or administration by an un-skilled care giver such as a family member. This can also be very important for acute episodes that require rapid intervention, such as migraine and other pain, anaphylaxis, seizure, and the like.

Portable drug delivery devices are preferred, as they can be carried by the user or care giver and be available when treatment is required. This feature can be very important for acute episodes that require rapid intervention, such as migraine and other pain, anaphylaxis, seizure, and the like.

Prefilled portable drug delivery systems, Prefilled self contained drug delivery systems, and portable, self contained drug delivery systems are particularly preferred. The most preferred drug delivery systems are prefilled, portable, and self contained. These systems are the most likely to have the best outcomes for a wide range of conditions, due to being easy to use, requiring minimal training, being small and discrete, being readily available when needed, requiring minimal steps for preparation and delivery, and reducing the amount of time skill required of a care giver. All of these features reduce time and cost of therapy, increase compliance, and increase positive outcomes.

A preferred embodiment of the drug delivery system is an autoinjector. Injection is preferred because of high bioavailability, reproducibility, ability to control and titrate dose, and rapid onset. Most pharmaceutically acceptable compounds can be injected, preferably in liquid form, although injection of solids and liquids is also known in the art.

A preferred embodiment of the autoinjector is the needle free injector. Needle free injectors are preferred because of:
No danger of needle stick injury and related exposure to disease
No needle phobia
Small diameter liquid jets result in little or no pain sensation
No requirement for sharps disposal
Very short flow path (as compared to a hypodermic needle) reduces viscous losses and enables delivery of high viscosity formulations.

Autoinjectors including needle free injectors can deliver any injection including intradermal, subcutaneous, intravenous, or intramuscular injections. Preferably, for the embodiment where the drug delivery system is an autoinjector, the injection is a sub-cutaneous injection.

In the most preferred embodiment, the drug delivery system is a prefilled, single dose, disposable, self contained, portable needle free injector comprising a borosilicate glass piston strengthened with ion exchange with a single injection orifice and a PTFE piston modified by the inclusion of less than 1% of PPVE and comprising two sealing ribs with the cross sectional shape of a frustrum.

Prefilled drug capsules must maintain container closure integrity over the labeled shelf life of the system. Preferred shelf lives include 1 year, preferably greater than one year, more preferably 2 years or more, most preferably 3 years or more. Container closure integrity must be maintained over the range of allowed storage temperatures, testing temperatures, and after sterilization of the components or terminal sterilization of the drug capsule. Storage, sterilization, and testing temperatures are preferably 15 to 30 degrees C., more preferably 2-40 degrees C., most preferably −10-50 degrees C., may be always above −10, 0, 2, 5, 10, 15, or 20 degrees C., and may be always below 100, 85, 75, 60, 50, 40, 30, or 25 degrees C.

FIG. 5 shows the results of a test of deformation of piston materials comparing PTFE to a PTFE modified by the inclusion of less than 1% by weight PPVE. After a 24 hours recovery from a 15 MPa load applied for 100 hours, it can be seen that the modified PTFE had significantly less deformation, 4% vs. 11% for the un-modified PTFE.

FIG. 6 shows how the resistance of PTFE modified with less than 1% PPVE to deformation under load is also seen at elevated temperatures.

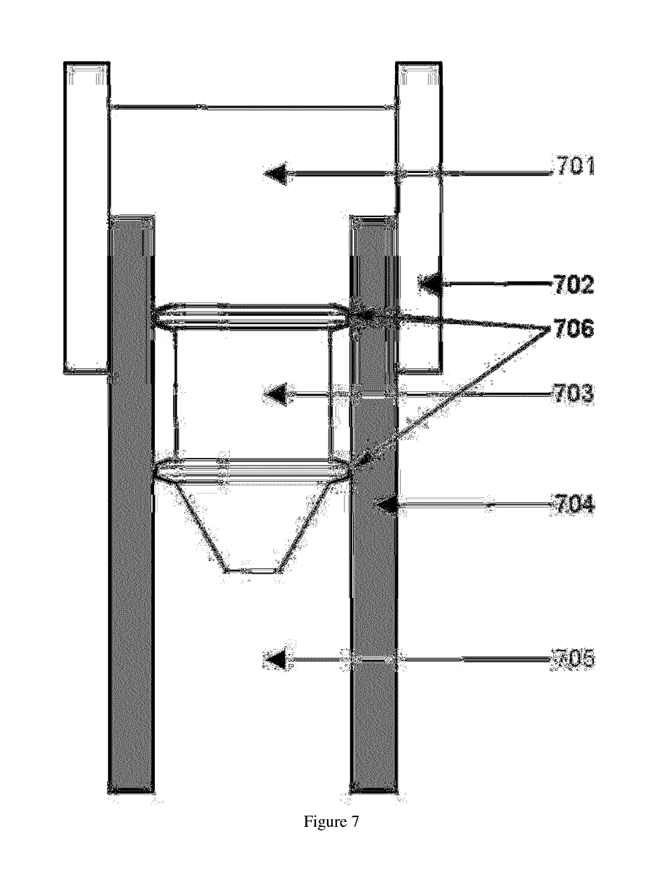

FIG. 7 shows schematically the apparatus used for dye ingress tests. Dye container 602 is placed sealingly about capsule 604, and is filled with dye 601. Liquid 605, usually normal saline, is contained within capsule 604. Piston 603 seals liquid 605 into capsule 604. Dye ingress is observed when the dye is seen to traverse one or both of the ribs of piston 603.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Drug capsules were constructed using borosilicate glass syringe bodies, and unmodified PTFE pistons. Before assembly the inside diameter of the syringe body and outside diameter of the piston ribs were measured and recorded. Twenty drug capsules were assembled and filled with normal saline.

Water-filled drug capsules were placed in an incubator and subjected to five thermal cycles between 40° C. and 2° C. The drug capsules were maintained for at least 12 hours at each temperature extreme. Following the thermal cycling, the pistons were subjected to a continuous dye ingress test for 24 hours at room temperature (20° C.).

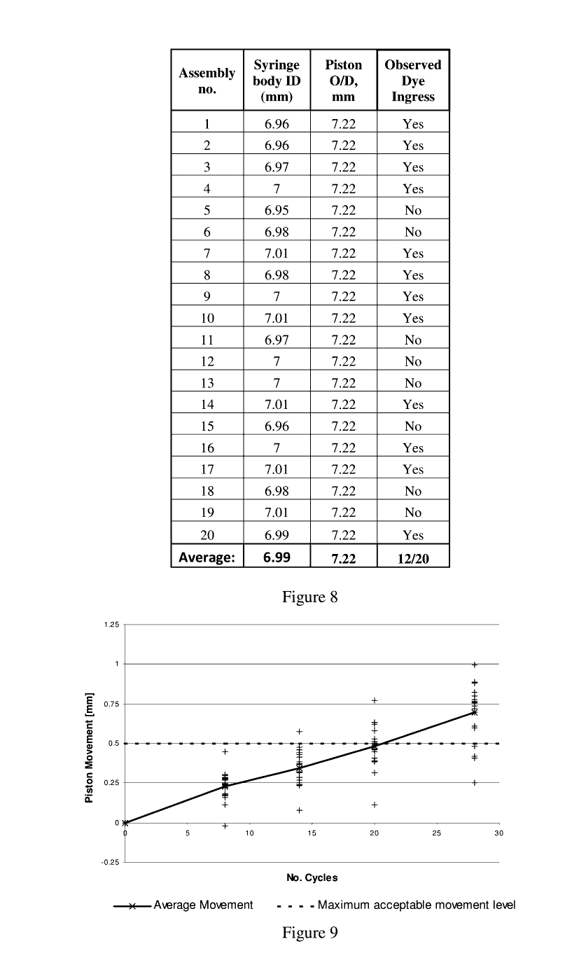

The results of the test are shown in FIG. 8. Notably, 12 of the drug capsules exhibited leakage, suggesting these capsules would have difficulty maintaining container closure integrity over the shelf life of the product.

### Example 2

20 drug capsules containing pistons made from glass filled PTFE were subjected to a thermal cycling test wherein they were cycled between 40° C. and 2° C. for 12 hours at each temperature for 30 days (i.e. 30 cycles). The piston movement was measured at regular intervals throughout the life cycle of the test. For this test, the maximum acceptable piston movement, based on previously determined requirements, was 0.5 mm

A graph of piston movement is shown in FIG. 9. As can be seen from this figure, the maximum acceptable movement was reached at 20 cycles, and was exceeded after 30 cycles.

### Example 3

Drug capsules were constructed using borosilicate glass syringe bodies, and modified PTFE pistons. The PTFE was modified by the introduction of less than 1% PPVE. Before assembly the inside diameter of the syringe body and outside diameter of the two piston ribs were measured and recorded. Twenty five drug capsules were assembled and filled with normal saline. The assembled drug capsules were then placed in an environmental chamber, and subjected to a 34 temperature cycles. Each cycle lasted one day and consisted of 12 hours at 40° C., followed by 12 hours at 2° C. After 8, 14, 20 and 34 cycles, the movement of the piston in the direction of the injection orifice was measured. Following the last cycle, the drug capsules were placed in a dye ingress apparatus (see FIG. 7) and tested for leakage.

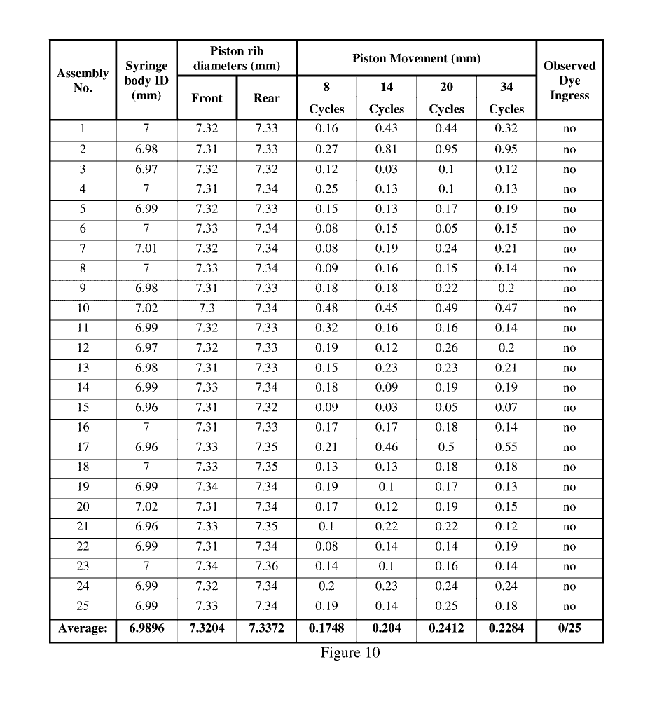

The results of these tests are shown in FIG. 10. Notably, as can be seen in the last column of FIG. 10, none of these cartridges exhibited leakage, leading to the expectation that cartridges assembled with pistons fabricated from this modified PTFE will maintain container closure integrity over the shelf life of the product. With a single exception, movement of the pistons did not exceed 0.5 mm, significantly better results than those seen with glass filled PTFE pistons, see example 2 above.

### Example 4

Drug cartridges were constructed using borosilicate glass syringe bodies, and modified PTFE pistons. The PTFE was modified by the inclusion of less than 1% PPVE, and differs from that presented in example 3 in that it had less PPVE to improve extrusion properties. Before assembly, the inside diameter of the syringe body and outside diameter of the two piston ribs were measured and recorded. Twenty cartridges were assembled and filled with normal saline. The assembled cartridges were then placed in an environmental chamber, and subjected to 29 temperature cycles. Each cycle lasted one day and consisted of 12 hours at 40° C., followed by 12 hours at 2° C. After 1, 4, 8, 12, 15, 21, and 29 cycles, the movement of the piston in the direction of the nozzle was measured. Following the last cycle, the cartridges were placed in a dye ingress apparatus (see FIG. 7) and tested for leakage.

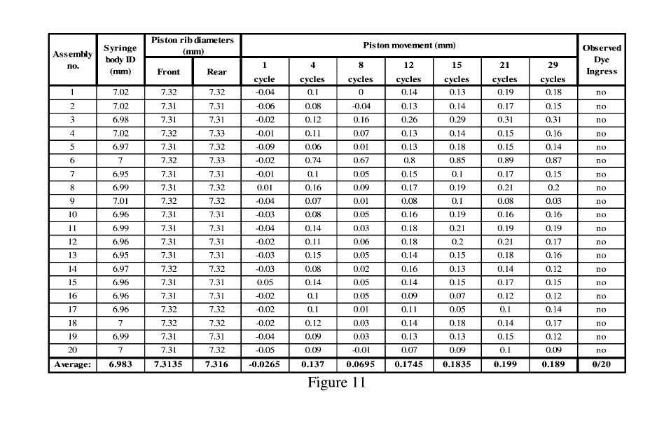

The results of these tests are shown in FIG. 11. Notably, as can be seen in the last column of FIG. 11, none of these cartridges exhibited leakage, leading to the expectation that cartridges assembled with pistons with this modified PTFE will maintain container closure integrity over the shelf life of the product. Again with only a single exception, movement of the pistons did not exceed 0.5 mm

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-20. (canceled)

21. A drug capsule for use in a drug delivery device, comprising:
a syringe body comprising borosilicate glass;
a piston comprising polytetrafluoroethylene (PTFE) contained within said syringe body;
wherein the PTFE has been modified by the inclusion of perfluor(propyl vinyl ether) (PPVE),
wherein the drug capsule is factory prefilled with a liquid formulation.

22. The drug capsule of claim 21 wherein the borosilicate glass is strengthened by ion exchange.

23. The drug capsule of claim 21, wherein the piston comprises less than 1% by weight of PPVE.

24. The drug capsule of claim 21 wherein the piston further comprises at least one circumferential rib of essentially triangular cross section with a triangle top where it contacts the syringe body removed.

25. The drug capsule of claim 21, wherein the drug capsule contains a drug formulation comprising a biologic drug further wherein the drug capsule does not contain a lubricant.

26. The drug capsule of claim 21, wherein the drug capsule is attached to an actuator to form a drug delivery system.

27. The drug capsule of claim 26, wherein the drug delivery system is an autoinjector with features comprising a characteristic selected from the group consisting of:
a spool valve;
a cap comprising a spin cap;
a spool retaining cage; and
a cap comprising two sets of threads.

28. The drug capsule of claim 26, wherein the drug delivery system comprises a characteristic selected from the group consisting of:
portable;
self-contained;
single dose disposable;
all mechanical,
being an autoinjector; and
being a needle free injector.

29. The drug capsule of claim 28, wherein the drug delivery system is a needle free injector.
wherein the needle free injector is portable, self-contained, single dose disposable, and all mechanical.

30. The drug capsule of claim 26, wherein the drug delivery system in a system selected from the group consisting of an injection system, a transdermal system, an inhalation system, an ocular system, a nasal system, a dermal system, and a buccal system.

31. The drug delivery system of claim 30.

32. The drug delivery system of claim 31, wherein the drug capsule comprises a biologically active agent selected from anti-inflammatory agents, antibacterial agents, antiparasitic agents, antifungal agents, antiviral agents, anti-neoplastic agents, analgesic agents, anaesthetics, vaccines, central nervous system agents, growth factors, hormones, antihistamines, osteoinductive agents, cardiovascular agents, anti-ulcer agents, bronchodilators, vasodilators, birth control agents and fertility enhancing agents, interferon alpha, growth hormone, osteoporosis drugs including PTH and PTH analogs and fragments, obesity drugs, psychiatric drugs, anti-diabetes, female infertility, AIDS, treatment of growth retardation in children, hepatitis, multiple sclerosis, and allergic reactions, hepatitis, multiple sclerosis, and allergic reactions.

33. The drug capsule of claim 32, wherein the drug capsule comprises a vaccine.

34. The drug capsule of claim 33, comprising a delivery orifice sealed by an end cap.

35. The drug capsule of claim 34, wherein the syringe body is contained in a polymeric sleeve.

36. The drug capsule of claim 34, wherein the polymeric sleeve comprises screw threads.

37. The drug delivery system of claim 26, wherein the actuator comprises a power source selected from:
a compressed gas charge;
batteries;
a mechanical spring; and
a pyrotechnic charge.

38. The drug delivery system of claim 37, further comprising:
an impact member which is movable to strike the piston and then continue to move the piston.

39. The drug delivery system of claim 37, wherein the actuator is triggered by pressing the drug delivery system against a desired injection site.

40. The drug delivery system of claim 37, comprising:
damping grease to damp recoil when the drug delivery system is pressed against a desired injection site.
